(19) United States
(12) Patent Application Publication
(10) Pub. No.: US 2017/0080132 A1
(43) Pub. Date: Mar. 23, 2017

(54) NASAL SUCTION DEVICE

(71) Applicant: Yuh-Jy Chen, New Taipei City (TW)

(72) Inventor: Ming-Chih Huang, New Taipei City (TW)

(21) Appl. No.: 15/018,717

(22) Filed: Feb. 8, 2016

(30) Foreign Application Priority Data
Sep. 21, 2015 (TW) 104131198

(51) Int. Cl. A61M 1/00; A61M 39/24

(52) U.S. Cl. CPC A61M 1/0003; A61M 1/0072; A61M 39/24; A61M 1/0086

(57) ABSTRACT

The present invention provides a nasal suction device, including a collection chamber, a resilient bulb, and a one-way valve. The collection chamber includes a main body, a cover, and an inner space, the cover being detachably connected to the main body in substantially airtight relation, to define the inner space. The resilient bulb is detachably connected to the main body in substantially airtight relation. The cover includes a first tube extending into the inner space, and the secretion passing by the first tube can be suctioned into the collection chamber. The main body includes a second tube, an opening of which adjacent to the resilient bulb is configured with the one-way valve.

### FIELD OF THE INVENTION

The present invention relates to a nasal suction device, in particular, to a manually nasal suction device.

### BACKGROUND OF THE INVENTION

The manual suction bulb of the conventional manually nasal suction device is usually applied as a container for the aspirated secretion as well, which causes the following issues: firstly, the common materials of the manual suction bulb are inappropriate to be high temperature sterilized, and the shape thereof causes difficulty for cleaning and drying it; secondly, when the manual suction bulb is repeatedly squeezed for aspiration, the secretion in the manual suction bulb or water vapor probably containing bacteria will be pumped out from the suction inlet.

Therefore, an improved nasal suction device is needed to overcome aforesaid issues in the related field.

### SUMMARY OF THE INVENTION

The present invention provides a nasal suction device which comprises a collection chamber, a resilient bulb and a one-way valve. The collection chamber includes a main body, a cover and an inner space.

The main body of the collection chamber includes a first end and a second end, the cover of the collection chamber includes a top end and a bottom end, and the inner space includes an upper part and a lower part. The cover is detachably connected to the main body in substantially airtight relation to define the inner space, wherein the bottom end of the cover is connected to the first end of the main body. The resilient bulb includes an open end and an air outlet, and the open end is detachably connected to the second end of the main body in substantially airtight relation.

In additional, the cover of the collection chamber includes a first tube extending into the inner space, and the secretion passing by the first tube can be suctioned into the collection chamber. The first tube includes a first opening at the top end of the cover, and a second opening at the lower end of the inner space.

The main body of the collection chamber includes a second tube, and the second tube includes a third opening at the second end of the main body, and a fourth opening at the upper of the inner space. The one-way valve is disposed on the third opening, allowing air flow from the inner space into the resilient bulb while blocking reverse air flow.

In another embodiment of the present invention, the first tube and the second tube are not coaxial.

According to the present invention, the material of the resilient bulb could be any resilient material which is suitable for achieving effect of manual suction bulb. In a specific embodiment of the present invention, the material of the resilient bulb can be silicone or rubber.

In another embodiment of the present invention, the nasal suction device further includes a suction head detachably connected to the first opening of the first tube.

In accordance with an embodiment of the present invention, the air outlet of the resilient bulb includes a chink. The chink forms an opening to pump air out when the resilient bulb is squeezed and deformed, and is closed to achieve an effect of substantially airtight when the resilient bulb is released form squeezing. In this embodiment, the material of the resilient bulb is preferred to be silicone or rubber.

In another embodiment of the present invention, the air outlet of the resilient bulb is a hole, a round hole for example. In this embodiment, when the resilient bulk is released from squeezing, a user can block the hole by finger to ensure the intensity of suction.

In another embodiment of the present invention, the material of the main body of the collection chamber is ABS resin, which is allowed to be high temperature sterilized.

In another embodiment of the present invention, the nasal suction device further includes a washer disposed between the main body and the cover.

The detail technical and preferred embodiments of the present invention are disclosed with the drawings as following, to make those of ordinary skill in the related field understand the features of the present invention.

### BRIEF DESCRIPTION OF THE DRAWINGS

By reading the present invention with the drawings, the foregoing summary and the detailed description of the present invention as following can be better understood. In the drawing:

FIG. 1 illustrates a schematic diagram in a perspective exploded view of a nasal suction device according to one embodiment of the present invention.

FIG. 2A illustrates a schematic diagram in a perspective view of a collection chamber according to the embodiment of the present invention, and FIG. 2B illustrates a schematic diagram in a cross-sectional view of the collection chamber according to the embodiment of the present invention; and

FIG. 3 illustrates a schematic diagram in an exploded and cross-sectional view of the collection chamber according to the embodiment of the present invention.

### DETAILED DESCRIPTION OF THE INVENTION

If not otherwise defined, all terms (including technical and scientific terms) has the same meaning as those of ordinary skill in the art understand.

As used herein, unless the context clearly dictates otherwise, the singular forms “a”, “an ” and “the present” include the plural forms (at least one).

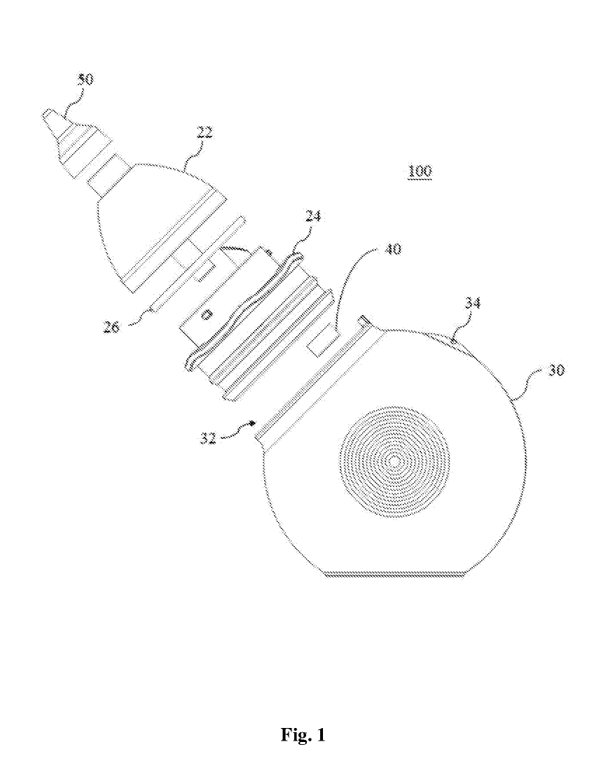

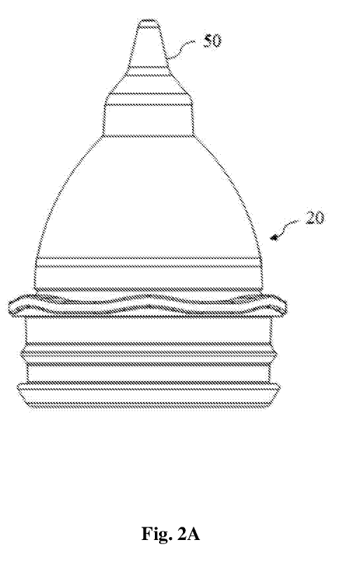

Referring to FIGS. 1 to 3, showing a nasal suction 100 of the present invention. The nasal suction device 100 of the present invention includes a collection chamber 20, a resilient ball 30 and a one-way valve 40. The collection chamber 20 includes a main body 24, a cover 22 and an inner space S 1. The main body 24 includes a first end 242 and a second end 244. The cover 22 includes a top end 222 and a bottom end 224, and the inner space S1 includes an upper part S12 and a lower part S14. The cover 22 is detachably connected to the main body 24 in substantially airtight relation to define the inner space S1, wherein the bottom end 224 of the cover 22 is connected to the first end 242 of the main body 24. The resilient bulb 30 includes an open end 32 and an air outlet 34, and the open end 32 is detachably connected to the second end 244 of the main body 24 in substantially airtight relation.

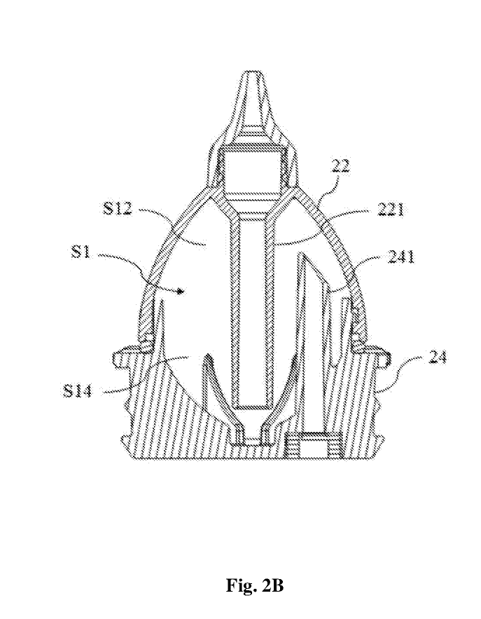

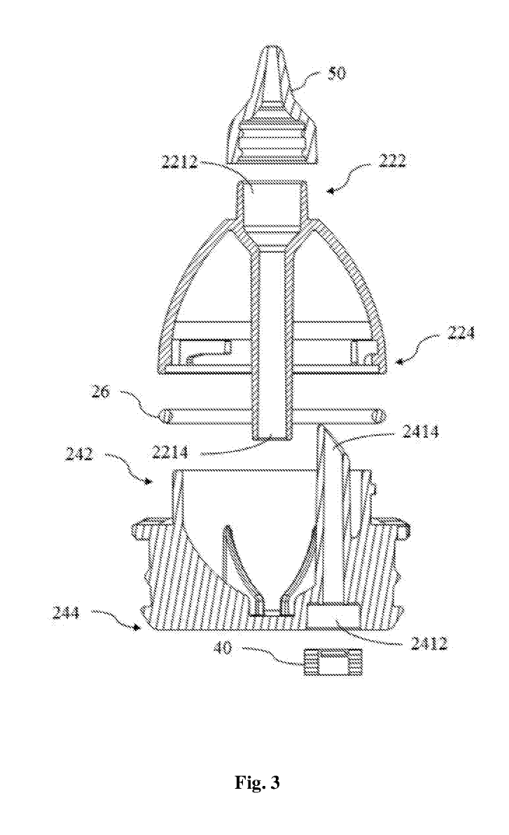

As shown in FIG. 2B and FIG. 3, the cover 22 includes a first tube 221 including a first opening 2212 at the top end 222 of the cover 22 and a second opening 2214 at the lower S14 of the inner space S1; the main body 24 includes a second tube 241 including a third opening 2412 at the second end 244 of the main body 24 and a fourth opening 2414 at the upper S12 of the inner space S1. The one-way valve 40 is disposed on the third opening 2412, allowing air flow from the inner space into the resilient bulb while blocking reverse air flow. In this embodiment, the first tube 221 and the second tube 241 are not coaxial.

Additionally, referring to FIGS. 1 to 3, in this embodiment, the nasal suction device 100 further includes a washer 26 and a suction head 50. The washer 26 is disposed between the main body 24 and the cover 22 to improve the airtight effect between the main body 24 and the cover 22. The suction head 50 is detachably connected to the first opening 2212 of the first tube 221. The suction head 50 can be replaceable by demand of users, such as different sizes of suction heads 50 for adult or kids.

Those of ordinary skill in the art can understand invention various modifications and alternative forms, the example shows by figure and here a detailed description of specific embodiments. Therefore, induction detailed description, it should be limited to any disclosed particular embodiments, but should cover all the variations and modifications are included within the scope of the present invention, as set forth in the following claims.

What is claimed is:

1. A nasal suction device comprising:
a collection chamber;
a resilient bulb; and
a one-way valve;
wherein the collection chamber comprises a main body comprising a first end and a second end, a cover comprising a top end and a bottom end and an inner space comprising an upper part and a lower part;
wherein the cover is detachably connected to the main body in substantially airtight relation to define the inner space;
wherein the bottom end of the cover is connected to the first end of the main body;
wherein the cover comprises a first tube having a first opening at the top end of the cover and a second opening at the lower end of the inner space, and the main body comprises a second tube having a third opening at the second end of the main body and a fourth opening at the upper part of the inner space;
wherein the resilient bulb comprises an open end and an air outlet, and the open end is detachably connected to the second end of the main body in substantially airtight relation; and
wherein the one-way valve is disposed on the third opening.

2. The nasal suction device according to claim 1, wherein the first tube and the second tube are not coaxial.

3. The nasal suction device according to claim 1, further comprising a suction head detachably connected to the first opening of the first tube.

4. The nasal suction device according to claim 1, wherein the air outlet of the resilient bulb comprises a chink.

5. The nasal suction device according to claim 1, wherein the air outlet of the resilient bulb comprises a hole.

6. The nasal suction device according to claim 1, wherein the material of the main body of the collection chamber comprises ABS resin.

7. The nasal suction device according to claim 1, wherein the material the resilient bulb comprises silicone or rubber.

8. The nasal suction device according to claim 1, further comprising a washer disposed between the main body and the cover.
